(19) United States
(12) Patent Application Publication
(10) Pub. No.: US 2016/0221270 A1
(43) Pub. Date: Aug. 4, 2016

(54) APPARATUS FOR MANUFACTURING DIAGNOSIS KIT, AND DIAGNOSIS KIT MANUFACTURED BY SAME

(71) Applicant: ACCESS BIO, INC., Somerset, NJ (US)

(72) Inventor: Seung Kook YU, Seoul (KR)

(73) Assignee: ACCESS BIO, INC., Somerset, NJ (US)

(21) Appl. No.: 15/021,248

(22) PCT Filed: Sep. 5, 2014
(86) PCT No.: PCT/KR2014/008370

§ 371 Date: Mar. 10, 2016

(30) Foreign Application Priority Data
Sep. 12, 2013 (KR) 10-2013-0109951

(51) Int. Cl. B29C 69/00; B29C 43/22; B29C 65/08; B65B 5/06; B65B 47/04; B65B 7/16; B65B 61/00; B65B 5/10; A61B 50/20; B29C 43/52; B65B 5/02

(52) U.S. Cl. CPC B29C 69/001; B29C 43/52; B29C 43/22; B29C 65/08; B65B 5/06; B65B 5/02; B65B 7/16; B65B 61/005; B65B 5/10; A61B 50/20; B65B 47/04; B29K 2105/256

(57) ABSTRACT

The present invention provides an apparatus for manufacturing a diagnosis kit, comprising: a molding member supplying unit; an upper molding unit for receiving an upper molding member from the molding member supplying unit and then molding the upper molding member into a predetermined upper diagnosis kit body; a lower molding unit for receiving a lower molding member from the molding member supplying unit and then molding the lower molding member into a predetermined lower diagnosis kit body, and inserting a diagnostic strip into the molded lower diagnosis kit body; a bonding unit for receiving an upper molding member and a lower molding member and then bonding the upper molding member and the lower molding member; and a cutting unit for cutting the bonded upper diagnosis kit body and lower diagnosis kit body into a predetermined diagnosis kit size.

### TECHNICAL FIELD

The present invention relates to an apparatus for manufacturing a diagnosis kit, and more particularly, an apparatus for manufacturing a diagnosis kit embedded with a diagnosis strip in a light-weight package, suitably configured for mass production, and a diagnosis kit manufactured thereby.

### BACKGROUND ART

A diagnosis kit is an apparatus to test or examine the existence of a single or a plurality of substances in a liquid sample, for example, a urine or blood sample.

More specifically, the modern diagnostic industry is falling into one type, POCT (Point-Of-Care Testing: POCT).

POCT is a test conducted outside of test laboratories and is a device that can be used by an ordinary person without professional knowledge. Presently, the use of POCT is spreading from hospitals to diagnostic usage in the fields and by individuals.

In particular, rapid diagnostic test kit, which is represented by immunochromatographic analysis, is used in healthcare and medical fields to identify diseases or changes, and various similar devices have been developed in various areas such as food and biological processes, environment technologies, etc., for quantitative and qualitative microanalysis. Its scope of application in the health care is also expanding such as into the field of pregnancy, ovulation, infectious diseases, drug abuse, acute myocardial infarction, and cancer.

Generally, such a diagnosis kit is formed of a case comprising a diagnostic strip according to the target diagnosis.

Here, the case of the diagnosis kit is formed by molding with a certain thickness.

These diagnosis kits are disposable because the diagnostic strip cannot be reused.

As such, the one-time use, disposable diagnosis kits manufactured by plastic molding result in waste of valuable resources.

In addition, manufacturing by molding also leads to increased volume of the diagnosis kits, which in turn require higher logistic cost for transportation.

As a prior reference related to the present invention, the Korean Patent Application Publication No. 10-2012-0086985 (published on Aug. 6, 2012) discloses a technology related to a method for manufacturing a diagnosis kit.

### DETAILED DESCRIPTION OF THE INVENTION

### Technical Objections

An aspect of the present invention is to provide an apparatus for manufacturing a diagnosis kit, in a large quantity, which can mold light-weight sheet or film containing a diagnostic strip inside of the sheet of film, and a diagnosis kit manufactured therewith.

Another aspect of the present invention is to provide an apparatus for manufacturing a diagnosis kit which can reduce the weight of the diagnosis kit and a diagnosis kit manufactured thereby.

Still another aspect of the present invention is to provide an apparatus for manufacturing a diagnosis kit, the apparatus can reduce the cost of manufacturing the diagnosis kit, facilitate design diversification, reduce the time period required for the development and preparation for mass production and cost of such development, and a diagnosis kit manufactured thereby.

### Means for Achieving the Technical Objection

In a preferable embodiment, the present invention provides an apparatus for manufacturing a diagnosis kit, comprising: a molding material feeding unit; an upper molding unit for receiving the upper molding material from the molding material feeding unit and molding it into an upper diagnosis kit body in a predetermined shape; a lower molding unit for receiving the lower molding material from the molding material feeding unit and molding it into a lower diagnosis kit body in a predetermined shape, and inserting a diagnostic strip in the lower diagnosis kit body; a bonding unit which receives the upper molding material molded in the upper molding unit and the lower molding material molded in the lower molding unit, and bonds the molded upper molding material and lower molding material in order to combine the upper diagnosis kit body and the lower diagnosis kit body; and a cutting unit for cutting the combined upper diagnosis kit body and the lower diagnosis kit body into a predetermined size of diagnosis kit.

The upper molding material and the lower molding material are preferable in the form of a sheet of film.

The upper molding unit is preferably comprised of an upper heating device for heating the upper molding material delivered from the upper material feeder, an upper press molding device for press molding the heated upper molding material into a predetermined shape of an upper diagnosis kit body, an upper cooling device for cooling down the press-molded upper molding material, and a punching device for forming a plurality of air holes in the diagnosis kit body included in the upper molding material.

The upper diagnosis kit body comprises an upper air tube protruding upwards in a rectangular circumference shape, a circular tube protruding upwards in a circular shape placed inside of the upper air tube, a test reagent injection hole formed inside of the circular tube, and a plurality of air holes formed inside of the air tube, with a certain distance from the circular tube.

The upper press molding device preferably press molds the upper air tube and the circular tube, and the punching device preferable punches the test reagent injection hole and the plurality of air holes at the same time.

The lower molding unit is preferably comprised of a lower heating device for heating the lower molding material delivered from the molding material feeder, a lower press molding device for press molding the heated upper molding material into a predetermined shape of a lower diagnosis kit body, a lower cooling device for cooling down the press-molded lower molding material, and a diagnostic strip inserting device for inserting a diagnostic strip in the lower diagnosis kit body.

The lower diagnosis kit body comprises a lower air tube having a size corresponding with that of the upper air tube, and a recessed hole formed inside of the air tube by a predetermined depth for seating the diagnostic strip.

The lower press molding device preferably press-molds the lower air tube and the recess.

In a configuration where the upper diagnosis kit body and the lower diagnosis kit body are formed in plurality,

The diagnostic strip inserting device preferably inserts a plurality of the diagnostic strips delivered in a row in the recessed holes formed in the lower diagnosis kit body sequentially or at the same time.

The bonding unit preferably heats and bonds the upper molded member and the lower molded member.

It is preferable that, with the bottom of the upper diagnosis kit body excluding the upper air tube and the circular tube contacting the upside of the lower diagnosis kit body excluding the lower air tube and the recess, both the two diagnosis kit bodies are heated to be fusion-bonded to form a body of the diagnosis kit.

The cutting unit preferable comprises: a cutting device for cutting out the diagnosis kit body from the fusion-bonded upper and lower molded members; a discharge device provided under the cutting device for dropping and discharging out the cut diagnosis kit body; and a collecting device for collecting the upper and lower molded member from which the diagnosis kit body is cut out.

In a different embodiment, the present invention provides a diagnosis kit manufactured using the apparatus for manufacturing a diagnosis kit in accordance with the present invention.

### Effect of the Invention

The present invention can manufacture diagnosis kits, in large quantities, by press-molding sheet or film containing diagnostic strips inside of the sheet of film.

In addition, the present invention can efficiently prevent deformation of kit itself by comprising a separate air tube on the diagnosis kit comprising a sheet or film material.

In addition, the present invention can reduce the weight of the diagnosis kits.

### BRIEF DESCRIPTION OF THE DRAWINGS

FIG. 1 shows an overall configuration in accordance with an embodiment of the present invention.

FIG. 2 is a view of the upper heating device in accordance with an embodiment of the present invention.

FIG. 3 is a perspective view of the upper diagnosis kit body in accordance with an embodiment of the present invention.

FIG. 4 is a top view of the upper diagnosis kit body in accordance with an embodiment of the present invention.

FIG. 5 is a cross-sectional view of FIG. 4 cut along the line A-A.

FIG. 6 is a view of the upper press molding device in accordance with an embodiment of the present invention.

FIG. 7 is a perspective view of the lower diagnosis kit body in accordance with an embodiment of the present invention.

FIG. 8 is a view of the lower press molding device in accordance with an embodiment of the present invention.

FIG. 9 is a cross-sectional view of FIG. 8 cut along the line B-B.

FIG. 10 is a view of the lower press molding device in accordance with an embodiment of the present invention.

FIG. 11 is a view of the diagnostic strip inserting device by a finger insertion type.

FIG. 12 is a view of the diagnostic strip inserting device by a vacuum gripper type,

FIG. 13 is a view of the diagnosis kit in accordance with an embodiment of the present invention, and

FIG. 14 is a cross-sectional view of the diagnosis kit of FIG. 13.

### THE BEST MODE FOR THE PRACTICE OF THE INVENTION

The manufacturing apparatus for diagnosis kit of the present invention is described below referring to the accompanying drawings.

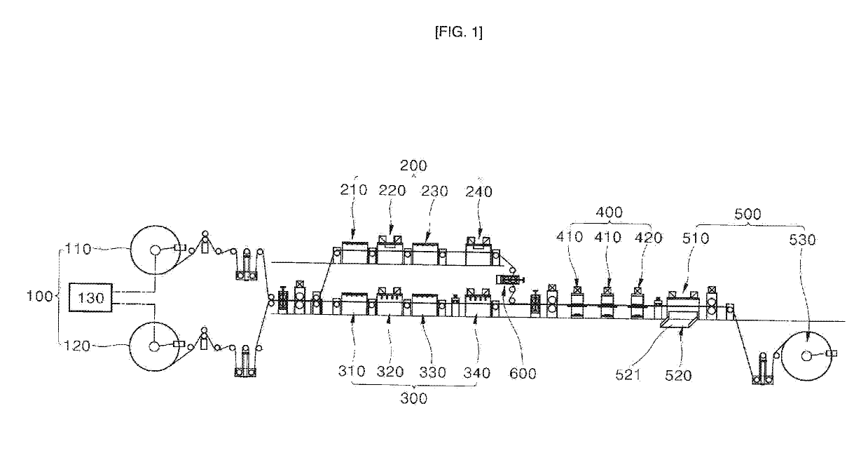

FIG. 1 shows an overall configuration in accordance with an embodiment of the present invention.

Referring to FIG. 1, the apparatus for manufacturing diagnosis kits in accordance with an embodiment of the present invention comprises a molding material feeding unit, an upper molding unit, a lower molding unit, a bonding unit, and a cutting unit.

Molding Material Feeding Unit (100)

In this description, the upper molding material (1) and the lower molding material (2) are supplied in a roll as a typical embodiment.

In addition, it will be obvious that the upper molding material (1) and the lower molding material (2) can also be supplied in a flat plate or slat form.

The molding material feeding unit (100) may comprise a first feed roll (11), a second feed roll (120), and a rotating device (130) e.g., an electric motor for driving the first and second feed rolls (110, 120).

The upper molding material (1) is wound on the first feed roll (110) and the lower molding material (2) is wound on the second feed roll (12).

Here, the upper molding material (1) and lower molding material (2) are formed in a sheet or film shape.

The upper and lower molding materials (1) and (2) can be any one of PA, APET, COPET, PET, PE, ABS, PS, PP, CPR, EG, PVC, PC, EVA, LDPE, and EVOH or a combination thereof.

In addition, the upper and lower molding materials (1) and (2) can be any material which can be heated and press molded.

A first feed path is provided for the upper molding material (1) between the first feed roll (110) and upper molding unit (200).

A second feed path is provided for the lower molding material (2) between the second feed roll (120) and lower molding unit (300).

A plurality of dancer rolls (D) are installed in the first feed path and the second feed path. The dancer rolls (D) apply appropriate tension to the upper and lower molding materials (1) and (2) to ensure stable moving along the first feed path and the second feed path.

Meanwhile, the upper molding material (1) and lower molding material (2) may be applied with an additional window forming process (not shown in the drawings) before they are wound on the first and second feed rolls (110) and (120).

The window is formed with a transparent material. The entire upper molding material (1) and lower molding material (2) can be formed with a transparent material or only a portion of the materials are provided with windows made of a transparent material.

Upper Molding Unit (200)

The upper molding unit (200) comprises an upper heating device (210), and upper press molding device (220), an upper cooling device (230), and a punching device (240).

The upper molding unit (200) provides a first molding path in connection with the first feed path.

In addition, the upper heating device (210), upper press molding device (220), upper cooling device (230), and punching device (240) are arranged along in the first molding path in said order.

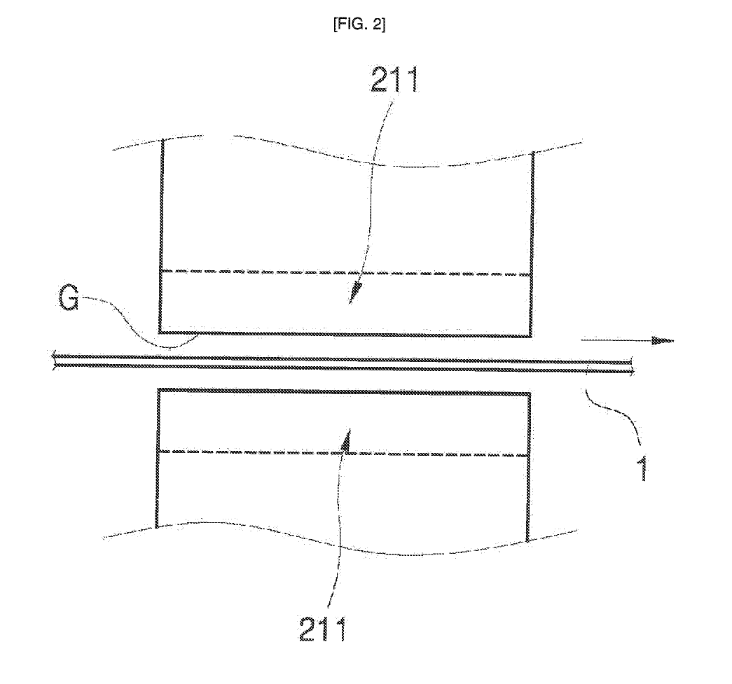

FIG. 2 is a view of the upper heating device in accordance with an embodiment of the present invention.

Referring to FIG. 2, the upper heating device (210) comprises a heating block (211) provided with a gap (G) through which the upper molding material (1) passes.

The heating block (211) is provided with a heater device (not shown) connected to an outside power source.

As such, the upper molding material (1) is heated by the heater device up to a predetermined temperature passing through the gap (G) of the heating block (211).

By the heating, the upper molding material (1) is changed to a press-moldable state.

In the present invention, the heating method can be a high frequency induction heating, radiative heating, or hot air heating.

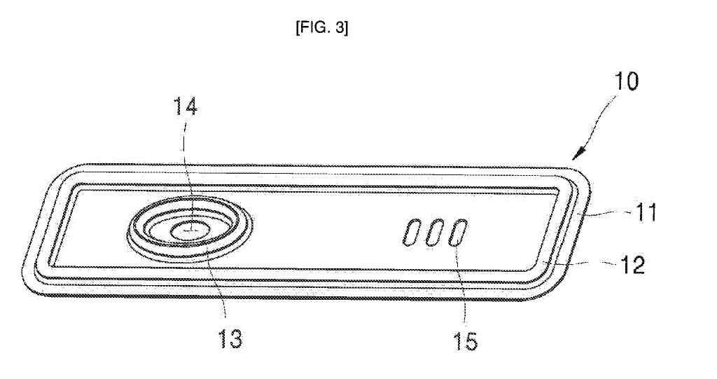

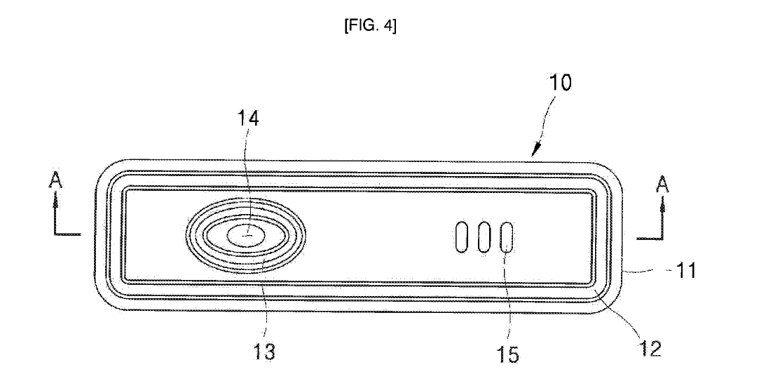

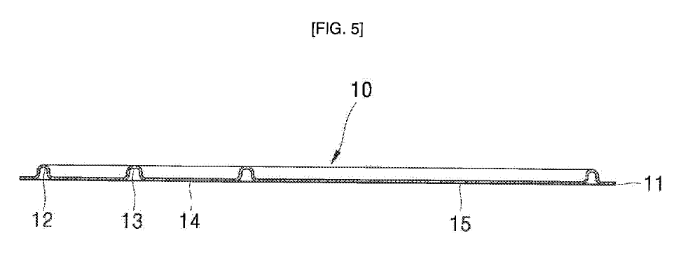

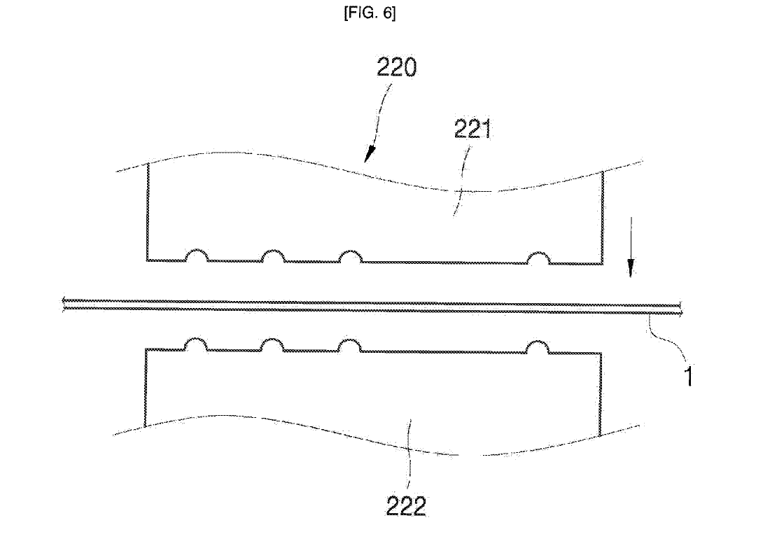

FIG. 2 is a view of the upper heating device in accordance with an embodiment of the present invention, FIG. 3 is a perspective view of the upper diagnosis kit body in accordance with an embodiment of the present invention, FIG. 4 is a top view of the upper diagnosis kit body in accordance with an embodiment of the present invention, FIG. 5 is a cross-sectional view of FIG. 4 cut along the line A-A, and FIG. 6 is a view of the upper press molding device in accordance with an embodiment of the present invention.

Next to the upper heating device (210) is an upper press molding device (220).

The upper press molding device (220) can be a press molding unit.

Referring to FIGS. 3 to 5, the configuration of the upper diagnosis kit body (10) in accordance with an embodiment of the present invention is described.

The upper diagnosis kit body (10) comprises: an upper body (11) formed with the upper molding material (1), un upper air tube (12) forming a rectangular circumference protruding upwards on the upper body (11), and a circular tube provided inside wall of the upper air tube (12).

In addition, a test reagent injection through-hole (14) is formed inside of the circular tube, and a plurality of air holes (15) are formed inside of the upper air tube (12) at a certain distance from the circular tube.

Referring to above described configuration, the upper press molding device (220) can comprise an upper mold (221) and a lower mold (222),and the upper molding material (1) is pressed and molded between the upper mold (221) and lower mold (222).

For example, referring to FIG. 6, the upper mold (221) is formed with the engraved patterns corresponding with the shapes of the upper air tube (12) and circular tube (13), and the lower mold (222) is formed with embossed patterns corresponding with the shapes of the upper air tube (12) and circular tube (13).

Accordingly, the upper molding material (1) is placed between the upper mold (221) and lower mold (222), and the upper mold (221) and lower mold (222) are pressed together to press-mold the upper molding material (1).

In addition, the test reagent injection hole (14) and punch holes (15) are formed with a separate punching device (240).

Here, any one or both of the upper mold (221) and lower mold (222) are provided with additional heating members (not shown), and any one or both of the upper mold (221) and lower mold (222) heated up by the heating members.

The upper cooling device (230) cools down the upper molding material (1) which is transferred after being formed with the upper air tube (12) and circular tube (13) by press molding described above, to a predetermined temperature level.

The cooling method can be water cooling or air cooling.

If an air cooling device is used, it is preferable to blow low cooling air on one side of the upper molding material (1) at a uniform pressure.

The punching device (240) is provided with a puncher (not shown) which moves up and down to punch holes.

Here, a plurality of punchers are provided to form test reagent injection hole (14) and air holes (15).

After cooling, the upper molding material (1) is fed into and placed at a predetermined position in the punching device (240).

The punch heads moves up and down to punch the test reagent injection hole (14) and air holes (15) in the upper molding material (1) placed at the predetermined position.

It should be noted that, the punching device (240) is separated from the upper cooling device (230) by a predetermined distance. The spacing allows the upper molding material (1) to be cooled down close to the room temperature so that the rims of the punch holes are not damaged.

Passing through the punching device (240), the upper molding material (1) is transferred to the arranging unit (600).

The method of preheating for heat forming can make use of hot air blow, a heating block, or a heating roll.

Lower Molding Unit (300)

The lower molding unit (300) comprises a lower heating device (310), a lower press molding device (320), a lower cooling device (330), and a diagnostic strip inserting device (340).

The lower molding unit (300) provides a second molding path in connection with the second feed path.

In addition, the lower heating device (310), lower press molding device (320), lower cooling device (330), and diagnostic strip inserting device (340) are arranged along in the second molding path in said order.

The lower heating device (310) comprises a heating block (311) provided with a gap (G) through which the lower molding material (2) passes.

The heating block (311) is provided with a heating member connected to an outside power source.

As such, the lower molding material (2) is heated by the heating member up to a predetermined temperature passing through the gap (G) of the heating block (311).

By the heating, the lower molding material (2) is changed to a press-moldable state.

In the present invention, the heating method can be a high frequency induction heating, radiative heating, or hot air heating.

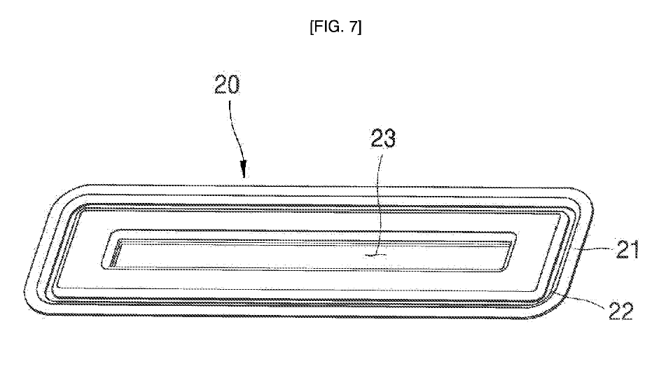

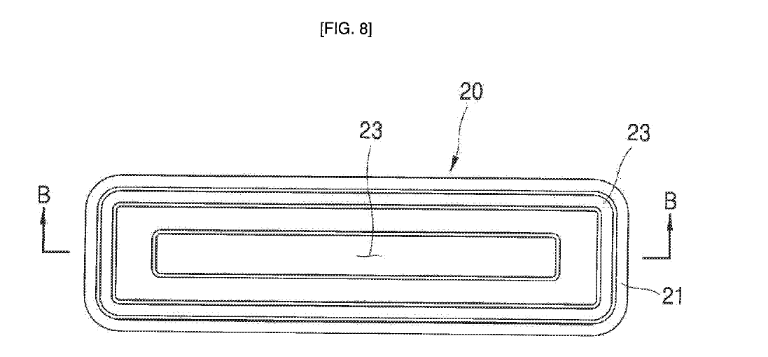

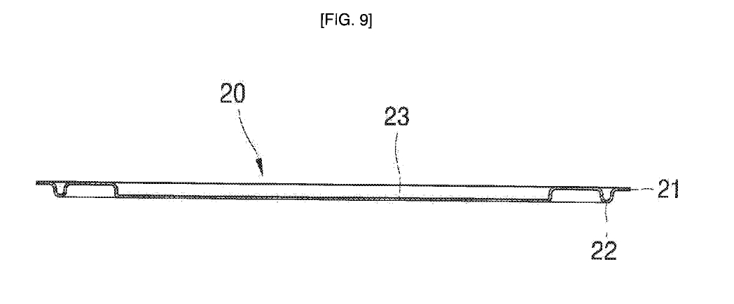

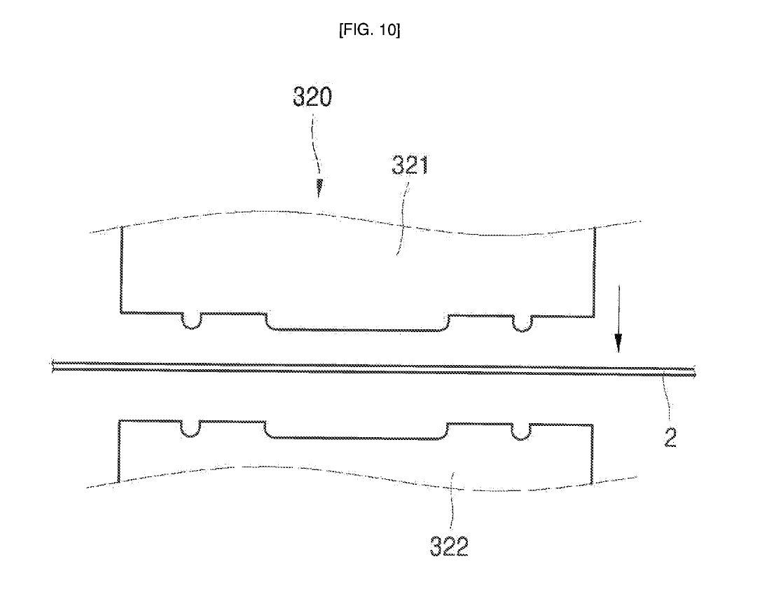

FIG. 7 is a perspective view of the lower diagnosis kit body in accordance with an embodiment of the present invention, FIG. 8 is a top view of the lower molding device in accordance with an embodiment of the present invention, FIG. 9 is a cross-sectional view of FIG. 8 cut along the line B-B, and FIG. 10 is a view of the lower press molding device in accordance with an embodiment of the present invention,

Next to the lower heating device (310) is a lower press molding device (320).

The lower press molding device (320) can be a press molding unit.

The configuration of a lower diagnosis kit body (20) in accordance with an embodiment of the present invention is described below.

The lower diagnosis kit body (20) comprises: a lower body (21) formed with the lower molding material (2), a lower body (20) convex downwards forming a rectangular circumference on the lower body (21), and a recessed hole (23) formed by a certain depth inside of the lower air tube (22).

Referring to above described configuration, the lower press molding device (320) can comprise an upper mold (321) and a lower mold (322),and the lower molding material (2) is pressed and molded between the upper mold (321) and lower mold (322).

For example, referring to FIGS. 7 to 9, the upper mold (321) is formed with embossed patterns corresponding with the shapes of the lower air tube (22) and recessed hole (23), and the lower mold is formed with engraved patterns corresponding with the shapes of the lower air tube (22) and recessed hole (23).

Accordingly, the lower molding material (2) is placed between the upper mold (321) and lower mold (322), and the upper mold (321) and lower mold (322) are pressed together to press-mold the lower molding material (2).

Here, the upper molding material (1) and lower molding material (2) can be formed with a plurality of upper diagnosis kit bodies (10) and lower diagnosis kit bodies (20) at a certain intervals.

Here, any one or both of the upper mold (221) and lower mold (222) are provided with additional heating members (not shown), and any one or both of the upper mold (221) and lower mold (222) heated up by the heating members.

In addition, while the present embodiment in accordance with the present invention is described as a press molding comprising an upper mold and a lower mold, it would be obvious for those skilled in the art that a roll press method can achieve the same performance.

Furthermore, the method can also be a heat molding, vacuum molding, or pressure forming.

The lower cooling device (330) cools down the lower molding material (2) which is transferred after being press-formed with the lower air tube (22) and recessed hole (23) described above, to a predetermined temperature level.

The cooling method can be water cooling or air cooling.

If an air cooling device is used, it is preferable to blow low cooling air on one side of the upper molding material (1) at a uniform pressure.

The diagnostic strip inserting device (340) inserts a diagnostic strip (30) in the recessed hole (23) formed in the lower diagnosis kit body (20) which has been cooled down.

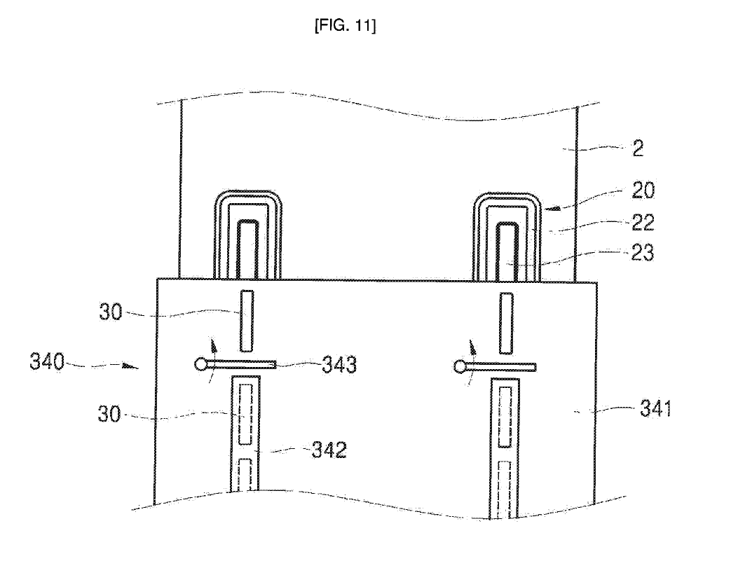

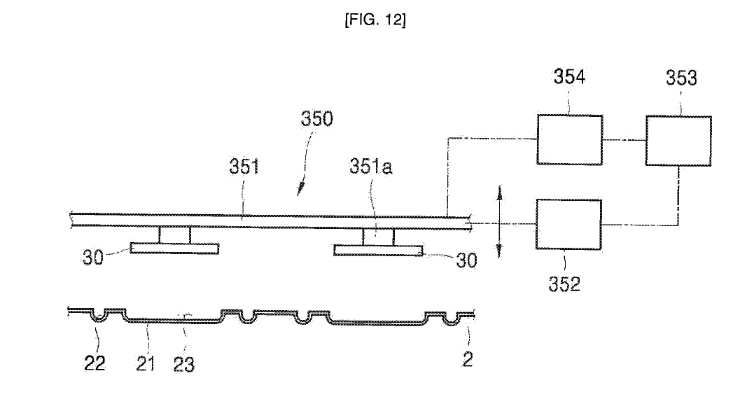

Diverse exemplary methods of inserting diagnostic strips in accordance with the present invention are described by referring to FIGS. 11 and 12.

FIG. 11 is a view of the diagnostic strip inserting device by a finger insertion type.

Referring to FIG. 11, the diagnostic strip inserting device (340) comprises: a strip loading device (342) which sequentially feeds diagnostic strips (30) to the base (341) and the feeding position formed on the base (341); and a motored swivel finger (343) which is provided on the lateral side of the feeding position and pushes the diagnostic strips (30) placed in the feeding position into the recessed hole (23).

Here, the base (341) is preferably positioned at a higher level than the second forming path.

Accordingly, when the press molded and cooled lower molding material (2) is transferred to the underside of the base (341), the swivel finger (343) rotates and pushes the diagnostic strip (30) placed at the feeding position into the recessed hole (22).

FIG. 12 is a view of a vacuum gripper type diagnostic strip inserting device.

Referring to FIG. 12, the diagnostic strip inserting device (350) comprises: a suction plate (351) having suction members (351a) for gripping a plurality of the diagnostic strips (30); a displacing device (352) for displacing the suction plate (351); and a vacuum controller (353) for generating vacuum pressure and controlling gripping.

Here, the spacing between the suction members (351 a) shall be substantially the same as the spacing between the recessed holes (23) formed on the lower molding material (2).

Accordingly, when the press-molded and cooled lower molding material (2) is transferred to the underside of the base (341), the suction plate (351) moves to the upside of the lower molding material (2) by the displacing device.

Here, the positions of the recessed holes (23) formed on the lower molding material (2) and the suction members (351a) are the same. The suction members (351a) are gripping the diagnosis kits by vacuum pressure.

The suction plate (351) is lowered by the displacing device (352) so that the diagnostic strips (30) are seated in the recessed holes (23), and the vacuum controller releases the vacuum pressure applied to the suction members (351a).

Accordingly, the vacuum strips (30) can be seated in the recessed holes (23).

While the method of inserting the diagnostic strips (30) was described above according to a typical method, any other methods which can seat the diagnostic strips (30) in the recessed holes (23) are applicable.

As described above, the lower molded material (2) seated with the diagnostic strips (30) is transferred to the arranging unit (600).

The preheating can be achieved by using hot air blow, a heating block, or a heating roll.

In an exemplary embodiment of the present invention described here, the upper molding material and the lower molding material are press molded, however, the upper molding material and the lower molding material can also be vacuum molded.

That is, while not depicted in the drawings, by contacting a body formed with desired patterns to the upper or lower surface of the preheated and moldable upper molding material and applying vacuum pressure to the desired patterns using an external vacuum suction device, the upper molding material can be vacuum molded in a desired shape.

In addition, the lower molding material can also be molded with the same vacuum molding method as the upper molding material.

Arranging Unit (600)

The arranging unit (600) in accordance with an embodiment of the present invention arranges the upper and lower molded materials (1) and (2) moving along the first and second molding paths, respectively, can be matched at a predetermined positions.

That is, it is preferable that the arrangement state shall be so that the upper air tube (12) formed on the upper molding material (1) and the lower air tube (22) formed on the lower molding material (2) are facing each other.

Bonding Unit (400)

Referring to FIG. 1, the bonding unit (400) in accordance with an embodiment of the present invention fusion-bonds the upper and lower molding materials (1) and (2) having passed the arranging unit (600) and contacting each other.

The bonding unit (400) provides a bonding path through which the contacting upper and lower molding materials (1) and (2) pass.

The bonding unit (400) comprises one or more heat bonding devices (410) and a cooling device which are arranged on the bonding path sequentially.

The one or more heat bonding devices (410) fusion-bonds the contacting surfaces of the upper and lower molding materials (1) and (2), sequentially, excluding the upper and lower air tubes (12) and (22).

The one or more heat bonding devices (410) can be ultrasonic welding devices, and their operation can be integrated into one step according to the dimension of the diagnosis kit to be manufactured.

In addition, the bonded material (1′) can be bonded by ultrasonic fusion.

The cooling device (420) cools down the bonded upper and lower molding members (hereinafter, “bonded material”).

Here, the bonded material (1′) is formed with a plurality of diagnosis kits.

Then, the cooled bonded material (1′) is transferred to the cutting unit (500).

In an embodiment of the present invention, the heat bonding device can use any one of heat fusion, high frequency induction fusion, and ultrasonic fusion.

Cutting Unit (500)

The cutting unit (500) cuts out the diagnosis kits from the bonding unit (400).

The cutting unit (500) is provided with a cutting device (510) having cutter knives (not shown) arranged corresponding with the circumference of the diagnosis kit, and cuts the circumference by moving up and down.

In addition, if the bonded material (1′) is formed with a plurality of diagnosis kits at a certain intervals, the cutting unit (500) may be provided with the cutter knives whose number corresponds with the number of the diagnosis kits.

Accordingly, a plurality of the diagnosis kits can be cut off at once by the cutter knives moving up and down.

The cutting unit (500) is provided with a discharge device (520) under the cutting device (510) for dropping and discharging the cutoff diagnosis kits.

The discharge device (520) is provided under the cutting device (510) and provided with a guide plate (521) for guiding the falling diagnosis kits.

In addition, the cutting unit (500) is provided with a collecting device (530) for collecting the bonded material (1′) excluding the diagnosis kits (hereinafter, “scrap”).

The collecting device (530) can be a collecting roller which winds and collects the bonded material (1′) from which the diagnosis kits have been cut off.

In the present invention, the scraps can be cut and processed.

The apparatus for manufacturing the diagnosis kits in accordance with an embodiment of the present invention is described above.

### Embodiments of the Present Invention

The diagnosis kit manufactured with the apparatus for manufacturing the diagnosis kits in accordance with an embodiment of the present invention is described in detail below.

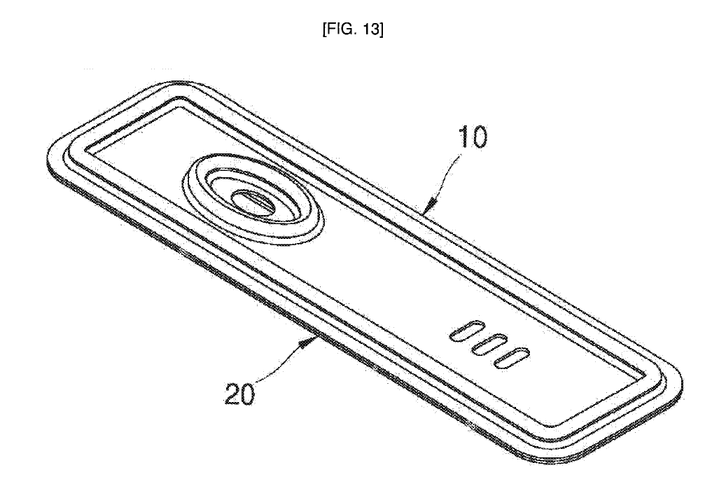

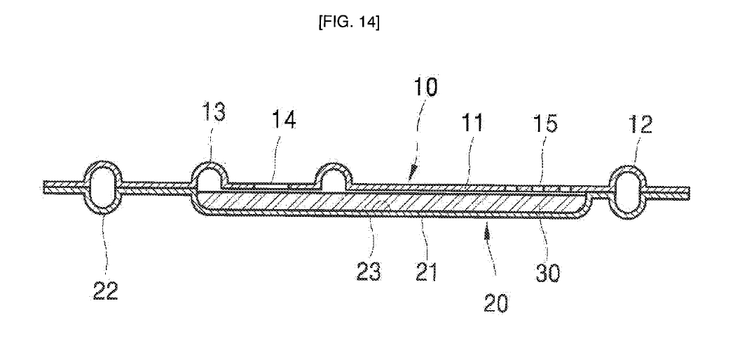

FIG. 13 is a view of the diagnosis kit in accordance with an embodiment of the present invention, and FIG. 14 is a cross-sectional view of the diagnosis kit of FIG. 13.

The configuration of the upper diagnosis kit body (10) and lower diagnosis kit body (20) will refer to FIGS. 3 and 7.

Referring to FIGS. 13 and 14, the diagnosis kit in accordance with an embodiment of the present invention comprises an upper diagnosis kit body (10) and a lower diagnosis kit body (20) which are fusion bonded with each other.

Upper Diagnosis Kit Body

The upper diagnosis kit body (10) is formed from a sheet or film material and provided with an upper body (11) which is a rectangular plate. It would be obvious for those skilled in the art that the the upper diagnosis kit body (10) can be formed in various shapes.

The upper body (11) is formed with an upper air tube (12) and a circular tube (13).

The upper air tube (12) forms a rectangular circumference on the upper body (11) and protrudes upward.

Under the upper body (11), the upper air tube (12) is formed with a space inside.

A purpose of the upper air tube (12) is to prevent the warping of the upper body (11).

Here, on the upper body (11), a circumference having a certain width is formed outside of the upper air tube (12).

In addition, the circular tube (13) is formed inside of the upper air tube (12) protruding upwards from the upper body (11).

Here, the longitudinal cross sections of the the upper air tube (12) and circular tube (13) can be the same.

The circular tube (13) also provides a support.

Furthermore, a test reagent injection hole (14) which is a vertical through-hole is formed inside of the circular tube (13).

And, a plurality of the air holes (15) are formed inside of the upper air tube (12) at a certain distance from the circular tube (13).

The plurality of air holes (15) are rectangular holes and formed as through-holes of the upper body (11).

Here, the formation of the upper diagnosis kit body (10) is described in the description of the apparatus for manufacturing, thus, not described here.

In addition, the upper air tube (12) and circular tube (13) can be formed in various shapes.

Lower Diagnosis Kit Body

The lower diagnosis kit body (20) is formed from a sheet or film material and provided with a lower body (21) formed in a rectangular planar-shape.

The lower body (21) is formed with an lower air tube (22) and a recessed hole (23).

The lower air tube (22) is formed in a rectangular circumference shape on the underside of the lower body (21) protruding downwards.

Here, the shape of the lower air tube (22) may be substantially the same as that of the upper air tube (12).

The recessed hole (23) is provided with a step or steps (s) at a certain depth on the upper surface of the lower body (21).

The size of the recessed hole (23) may be determined variably in accordance with the size of the diagnostic strip (30) to be seated in the recess.

Here, since the description of the formation of the lower diagnosis kit body (20) is described in the description of the apparatus for manufacturing, thus, omitted.

In accordance with an embodiment of the present invention, the upper diagnosis kit body (10) and the lower diagnosis kit body (20) are contacted and bonded with each other, and the method of bonding is preferably heat fusion as described above.

That is, because the upper and lower diagnosis kit bodies (10 and 20) are formed from a sheet or film material, they can be fusion bonded.

Accordingly, the upper body (11) and the lower body (21) form an integral body and the upper and lower air tubes are formed protruding upwards and downwards, respectively.

In addition, the lower air tube (22) can be formed in various shapes.

And, top of the recessed hole (23) having a certain depth and length is covered by the upper body (11).

Here, the recessed hole (23) is seated with a diagnostic strip (30).

The diagnostic strip (30) is a test material that can diagnose various diseases.

Here, an end of the diagnostic strip (30) is exposed to the test reagent injection hole (14) formed in the upper body (11) while another end is exposed to the plurality of air holes (15).

As such, an end of the diagnostic strip is smeared with the test reagent entering through the test reagent injection hole, and the test reagent can be easily transferred to the opposite end by the air penetrating into the air holes on the opposite end.

The exemplary embodiments of the apparatus for manufacturing a diagnosis kit and the diagnosis kit manufactured thereby in accordance with the present invention are described above, it would be obvious that various modifications and changes can be made without departing from the spirit of the present invention.

The present invention may be embodied in other specific forms without departing from the spirit or essential attributes thereof and accordingly, reference should be made to the appended claims rather than to the foregoing specification as indicating the scope of the invention

Therefore, the embodiments described here shall be interpreted to be exemplary and not limiting the present invention. It will be understood that the appended claims are intended to cover all such modifications and embodiments which come within the spirit and scope of the present invention.

1. An apparatus for manufacturing a diagnosis kit, comprising: a molding material feeding unit for feeding upper molding material and lower molding material via independent feeding paths; an upper molding unit for receiving the upper molding material from the molding material feeding unit to mold it into an upper diagnosis kit body in a predetermined shape; a lower molding unit for receiving the lower molding material from the molding material feeding unit to mold it into a lower diagnosis kit body in a predetermined shape, and inserting a diagnostic strip in the lower diagnosis kit body; a bonding unit for receiving the upper molded material from the upper molding unit and the lower molded material from the lower molding unit and bonding them so that the upper diagnosis kit body and the lower diagnosis kit body are bonded with each other; and a cutting unit for cutting the bonded upper diagnosis kit body and lower diagnosis kit body into a diagnosis kit having a predetermined size.

2. The apparatus of claim 1, wherein the upper molding material and the lower molding material are substantially a sheet or film material.

3. The apparatus of claim 2, wherein the upper molding unit comprises: an upper heating device for heating the upper molding material fed from the molding material feeding unit to make it moldable; an upper press molding device for press molding the upper molding material heated and moldable into an upper diagnosis kit body; an upper cooling device for cooling the press-molded upper molding material; and a punching device for punching an air hole in the upper diagnosis kit body.

4. The apparatus of claim 3, wherein the upper diagnosis kit body comprises: an upper air tube formed in a rectangular circumference shape protruding upwards; a circular tube formed inside of the upper air tube in a circular circumference shape protruding upwards; a test reagent injection hole formed inside of the circular tube; and a plurality of air holes formed inside of the air tube at a certain distance from the circular tube, wherein the upper press molding device press molds the upper air tube and the circular tube, and the punching device punches the test reagent injection hole and the plurality of air holes at the same step.

5. The apparatus of claim 4, wherein the lower molding unit comprises: a lower heating device for heating the lower molding material fed from the molding material feeding unit to make it moldable; a lower press molding device for press molding the lower molding material heated and moldable into a lower diagnosis kit body; a lower cooling device for cooling the press-molded lower molding material; and a diagnostic strip inserting device form inserting diagnostic strip in the lower diagnosis kit body.

6. The apparatus of claim 5, wherein the lower diagnosis kit body is formed with a lower air tube having a size corresponding with the size of the upper air tube, and a recessed hole formed inside of the lower air tube by a certain depth for receiving the diagnostic strip, and the lower press molding device forms the lower air tube and the recessed hole by press molding.

7. The apparatus of claim 6, if the upper diagnosis kit body and the lower diagnosis kit body are formed in a plurality, the diagnostic strip inserting device inserts the diagnostic strips in place into the recessed holes formed in the lower diagnosis kit body sequentially or all at once.

8. The apparatus of claim 6, wherein the bonding unit receives the upper molded material and the lower molded material by heat fusion bonding, contacting the bottom of the upper diagnosis kit body excluding the upper air tube and the circular tube and the upside of the lower diagnosis kit body excluding the lower air tube and the recessed holes, and bonding the contact surface by heating to form a diagnosis kit body.

9. The apparatus of claim 8, wherein the cutting unit comprises: a cutting device for cutting out the diagnosis kit body from the bonded upper and lower molded materials; a discharging device for dropping and discharging the cut-out diagnosis kit body; and a collecting device for collecting the upper and lower molded materials from which the diagnosis kit has been cut off.

10. A diagnosis kit manufactured with the apparatus for manufacturing diagnosis kits of claim 1.
